(19) United States
(12) Patent Application Publication
(10) Pub. No.: US 2012/0041173 A1
(43) Pub. Date: Feb. 16, 2012

(54) WATER SOLUBLE SOLID PHASE PEPTIDE SYNTHESIS

(75) Inventor: Jonathan M. Collins, Charlotte, NC (US)

(73) Assignee: CEM CORPORATION, Matthews, NC (US)

(21) Appl. No.: 13/209,960

(22) Filed: Aug. 15, 2011

Related U.S. Application Data
(60) Provisional application No. 61/373,989, filed on Aug. 16, 2010.
(60) Provisional application No. 61/382,550, filed on Sep. 14, 2010.
(60) Provisional application No. 61/441,390, filed on Feb. 10, 2011.
(60) Provisional application No. 61/469,881, filed on Mar. 31, 2011.

(51) Int. Cl. C07K 1/04; C07K 1/10; C07K 2/00

(52) U.S. Cl. 530/334; 530/300; 204/157.68

(57) ABSTRACT

A solid phase peptide synthesis method is disclosed. The method includes the steps of deprotecting an amino group in its protected form that is protected with a protecting group containing a Michael acceptor site composed of an α,β-unsaturated sulfone in a solvent selected from the group consisting of water, alcohol, and mixtures of water and alcohol; washing the deprotected acid in a solvent selected from the group consisting of water, alcohol, and mixtures of water and alcohol; coupling the deprotected acid to a resin-based peptide or a resin-based amino acid in a solvent selected from the group consisting of water, alcohol, and mixtures of water and alcohol; and washing the coupled composition in a solvent selected from the group consisting of water, alcohol, and mixtures of water and alcohol.

### RELATED APPLICATIONS

This application claims priority from U.S. provisional application Ser. Nos. 61/373,989 filed Aug. 16, 2010; 61/382,550 filed Sep. 14, 2010; 61/441,390 filed Feb. 10, 2011 and 61/469,881 filed Mar. 31, 2011.

### BACKGROUND

The present invention relates to solid phase peptide synthesis (SPPS) and to a method of carrying out SPPS reactions in aqueous solutions.

Peptides are linked chains of amino acids which in turn are the basic building blocks for most living organisms. Peptides are also the precursors of proteins; i.e., long complex chains of amino acids. Peptides and proteins are fundamental to human and animal life, and they drive, affect, or control a wide variety of natural processes. As a result, the study of peptides and proteins and the capability to synthesize peptides and proteins are of significant interest in the biological sciences and medicine.

Solid phase peptide synthesis is a technique in which an initial amino acid is linked to a solid particle and then additional amino acids are added to the first acid to form the peptide chain. Because the chain is attached to a particle, it can be washed and otherwise treated with additional solvents or rinses while being maintained in a discrete vessel and handled (at least to some extent) as a solid. SPPS thus allows solution phase chemistry to be carried out in a manner that has some of the convenience of handling solids.

Conventional SPPS is most typically carried out in polar organic solvents such as dimethyl formamide (DMF), n-methylpyrrolidone (NMP), dimethyl sulfoxide (DMSO) and dichloromethane (DCM). DCM is typically mixed with DMF or NMP because the N-alpha protecting groups Fmoc (e.g., fluorenylmethyloxycarbonyl chloride) and Boc (e.g., tert-butoxycarbonyl) frequently used in SPPS are typically hydrophobic and insoluble in water. Although Fmoc and Boc (e.g., tert-butoxycarbonyl) synthesis methods have had a major impact on SPPS they both suffer from their need for organic solvents that are costly and toxic.

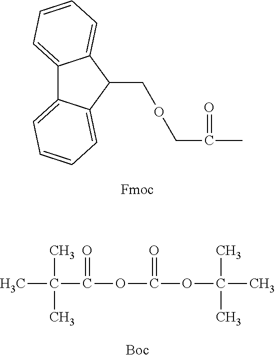

These toxic solvents require the use of special laboratory techniques, such as carrying out the reactions entirely under a fume hood or equivalent device. Fume Hood space is limited and thus valuable in the laboratory context. As a result, SPPS using these solvents is expensive from a landscape standpoint.

These organic solvents tend to be aggressive and require upgraded equipment. Their disposal represents an environmental hazard and at a minimum is regulated.

In conventional SPPS, the Fmoc group is removed by a secondary amine (piperidine, piperazine, morpholine) in a β-elimination reaction during SPPS. An undesirable feature of this mechanism is that it generates a reactive dibenzofulvene (DBF) that is scavenged by excess piperidine. The DBF can, however, also react with the free amine group effectively capping the end of the peptide chain. Some deprotection employ a short initial deprotection step to flush most of the DBF out of the reaction vessel and then use a second longer deprotection with fresh piperidine solution to reduce this potential side reaction. This approach may be unnecessary, however, because a typical 20% deprotection solution has a large excess of piperidine versus potential DBF. For example, a synthesis at 0.1 mmol scale using a 7 mL solution of a 20% piperidine in DMF would have a ratio of piperidine to total potential DBF of approximately 710:1.

Based upon these and other factors, an aqueous based—i.e., water-soluble—scheme for peptide synthesis, and particularly SPPS, represents a worthwhile ongoing technological goal.

As one attempt, some authors have hinted that finely powdered or pulverized reagents can increase the water solubility of the relevant SPPS compositions, but such results are to date difficult to confirm or reproduce.

As another attempt, Galanis (Organic Letters, Vol. 11, No. 20, pp. 4488-4491 (2009)) has used a conventional Boc protecting group in the presence of specific resins, linkers, activating agents and a zwitterion detergent to produce a single demonstrative Leu-Enkephalin peptide.

As a more promising option, water soluble protecting groups have been attempted. Hojo (Hojo et al; Chem. Pharm. Bull. 52, 422-427 2004; Hojo, K.; Maeda, M.; Kawasaki, K. Tetrahedron Lett. 45, 9293 2004) has developed several protecting groups for this purpose that include 2-(Phenyl(methyl)sulfoniol)ethyloxy carbonyl tetrafluoroborate (Pms), Ethanesulfonylethoxycarbonyl (Esc), and 2-(4-Sulfophenylsulfonyl)ethoxy carbonyl (Sps).

These reports are, of course, exemplary rather than comprehensive.

Although amino acids carrying these protecting groups are water-soluble, the groups raise other difficulties that make their routine use more difficult. The Pms group is an onium salt and thus significantly less stable than conventional protecting groups. Esc is more stable than Pms and offers moderate aqueous solubility. The starting material, however, for the Esc group is relatively expensive. Additionally, the Esc-Cl group is unstable and the group must be converted to ethanesulfonylethyl-4-nitrophenyl carbonate (ESC-ONp) for use with amino acids.

Sps has a solubility comparable to that of Esc, but synthesizing Esc appears to be more complicated and expensive. Additionally, a different synthesis scheme must be used for cysteine (Cys) and methionine (Met) in order to avoid oxidation of their sulfur groups.

As a secondary consideration, a larger number of aromatic rings in a protecting group molecule can enhance the UV absorption for conventional monitoring purposes. The additional rings, however, also minimize or eliminate water solubility.

In conventional monitoring methods, a reaction product is drawn after the deprotection step and measured under UV absorption. Fmoc will absorb characteristic UV frequencies (e.g., 300 nanometers) in amount proportional to its concentration and thus the amount of detected Fmoc will provide an indication of the extent to which deprotection has proceeded

Because of their molecular structure, Pms, Esc, and Sps have the advantage of some water solubility, but Pms and Esc cannot be tracked in conventional UV monitoring in the same manner as conventional Fmoc. Sps can be monitored by UV, but its difficult and costly synthesis tends to discourage its use. As a result, the increased water solubility of these compounds is less helpful in an overall sense.

Therefore, a need continues to exist for improved water soluble (aqueous-based) reaction systems for peptide synthesis in general and solid phase peptide synthesis in particular.

### SUMMARY

The invention is an improvement in solid phase peptide synthesis. In a broad aspect, the invention includes the steps of deprotecting an amino acid that is soluble in water in its protected form and that is protected with a protecting group that acts as a Michael Reaction acceptor in the presence of a Michael Reaction donor, and washing the deprotected acid in a solvent selected from the group consisting of water, alcohol, and mixtures of water and alcohol.

In exemplary aspects, the invention includes the steps of deprotecting an amino group in its protected form that is protected with a protecting group containing a Michael acceptor site composed of an alpha, beta (α,β) unsaturated sulfone and then washing the deprotected acid in a solvent selected from the group consisting of water, alcohol, and mixtures of water and alcohol.

In exemplary aspects, the protecting group is selected from the group consisting of Bsmoc, Nsmoc, Bspoc and Mspoc; and with Bsmoc being typical.

In another aspect, the invention is a solid phase peptide synthesis method that includes the improvement of deprotecting a Bsmoc-protected amino acid, and then washing the deprotected acid in a solvent selected from the group consisting of water, alcohol, and mixtures of water and alcohol.

In another aspect, the invention is a solid phase peptide synthesis method that includes the improvement of deprotecting an amino group in its protected form that is protected with a protecting group containing a Michael acceptor site composed of an α,β-unsaturated sulfone in a solvent selected from the group consisting of water, alcohol and mixtures of water and alcohol

In another aspect, the invention is a solid phase peptide synthesis method that includes the improvement of deprotecting an amino group in its protected form that is protected with a protecting group containing a Michael acceptor site composed of an α,β-unsaturated sulfone, and then coupling the deprotected acid to a resin-based peptide or a resin-based amino acid in a solvent selected from the group consisting of water, alcohol, and mixtures of water and alcohol

In another aspect, the invention is a solid phase peptide synthesis method that includes the steps of deprotecting an amino group in its protected form that is protected with a protecting group containing a Michael acceptor site composed of an α,β-unsaturated sulfone in a solvent selected from the group consisting of water, alcohol, and mixtures of water and alcohol; washing the deprotected acid in a solvent selected from the group consisting of water, alcohol, and mixtures of water and alcohol; coupling the deprotected acid to a resin-based peptide or a resin-based amino acid in a solvent selected from the group consisting of water, alcohol, and mixtures of water and alcohol; and washing the coupled composition in a solvent selected from the group consisting of water, alcohol, and mixtures of water and alcohol.

In another aspect, the invention is a composition that includes a mixture of a solid phase resin and a solution. The solution comprises an amino acid and an amino acid protecting group, both dissolved in the same solvent. The protecting group contains a Michael acceptor site composed of an α,β-unsaturated sulfone, and the solvent is selected from the group consisting of water, alcohol, and mixtures of water and alcohol.

In another aspect, the invention is a process for accelerating the solid phase synthesis of peptides. In this aspect, the invention includes the steps of deprotecting the alpha-amino group of a first an amino group in its protected form that is protected with a protecting group containing a Michael acceptor site composed of an α,β-unsaturated sulfone and linked to solid phase resin particles by admixing the protected linked acid with a deprotecting solution in a microwave transparent vessel while irradiating the admixed acid and solution with microwaves; activating a second amino acid by adding the second acid and an activating solution to the same vessel; coupling the second amino acid to the first acid while irradiating the composition in the same vessel with microwaves; and successively deprotecting, activating, and coupling a plurality of amino acids into a peptide in the same microwave transparent vessel without removing the peptide from the same vessel between cycles.

### DETAILED DESCRIPTION

In a broad aspect, the invention is a solid phase peptide synthesis method in which the improvement comprises deprotecting an amino acid that is soluble in water in its protected form and that is protected with a protecting group that acts as a Michael Reaction acceptor in the presence of a Michael Reaction donor in a solvent selected from the group consisting of water, alcohol, and mixtures of water and alcohol.

In another aspect, the invention is a solid phase synthesis method in which the improvement comprises deprotecting an amino group in its protected form that is protected with a protecting group containing a Michael acceptor site composed of an, unsaturated sulfone, and washing the deprotected acid in a solvent selected from the group consisting of water, alcohol, and mixtures of water and alcohol.

In exemplary aspects, the protecting group is selected from the group consisting of Bsmoc, Nsmoc, Bspoc and Mspoc; and with Bsmoc being typical.

As well understood by the skilled person, a Michael Addition reaction is the nucleophilic addition of a nucleophile to an alpha, beta unsaturated carbonyl compound. The nucleophile is the Michael Donor (e.g., piperidine) and the alpha, beta unsaturated carbonyl compound is the Michael Acceptor (e.g. an alkene).

In exemplary embodiments of the present invention, the amino acid protecting group has a Michael acceptor site that includes an alpha, beta-unsaturated sulfone.

As discussed in detail herein, such a compositions include (but are not necessarily limited to) compounds that are abbreviated herein as Bsmoc, Nsmoc, Bspoc and Mspoc.

It will also be understood that as used herein, a phrase such as “soluble in water in its protected form” means that the composition has the degree of solubility necessary for the desired reaction to proceed in an aqueous solvent system. As is the case with any composition, the term “soluble” does not imply unlimited solubility in any or all amounts.

In another aspect, the acid is protected with Bsmoc, and is deprotected in a solvent selected from the group consisting of water, alcohol, and mixtures of water and alcohol. As used herein, the abbreviation Bsmoc refers to 1,1-dioxobenzo[b]thiphene-2-ylmethyloxycarbonyl. Bsmoc is also referred to by the “common name” benzo[b]thiophenesulfone-2-methyloxycarbonyl. Bsmoc is typically represented by the following formula:

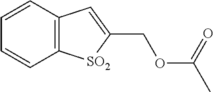

An early discussion of Bsmoc as a protecting group for amino acids during SPPS synthesis is set forth by Carpino et al in the Journal or Organic Chemistry, 1999, 64 (12) at pages 4324-4338.

Four of the standard Bsmoc amino acid derivatives are difficult to handle at room temperature [Bsmoc-Asp(OtBu)—OH, Bsmoc-Leu-OH, Bsmoc-Pro-OH, Bsmoc-Ser(tBu)—OH] because they are either oils or have a low melting point (Asp—m.p.˜43° C.). The 16 other Bsmoc derivatives are solids at room temperature with melting temperatures greater than 90° C. Therefore, for the four Bsmoc derivatives that are more difficult to handle the use of a higher molecular weight derivative Nsmoc (e.g., 1,1-dioxonaptho[1,2-b]thiophene-2-methyloxycarbonyl; “α-Nsmoc”) is recommended.

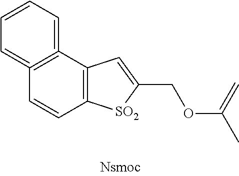

Nsmoc derivatives of all 20 standard amino acids have been successfully made and used in SPPS. The Nsmoc group shows similar advantages to the Bsmoc group, but appears somewhat more expensive to produce because of its additional six member carbon ring. The Nsmoc group is also predicted to result in a lower acylation rate than the Bsmoc group, but comparable to the Fmoc group because of their similar size. As a further possibility (and as known to the skilled person), two other Nsmoc isomers can be produced; i.e., with the second aromatic ring in a different position with respect to the SO₂ group.

Related protecting groups that can function as the Michael acceptor include 2-tert-butylsulfonyl-2-propenoxycarbonyl (Bspoc) and 2-methylsulfonyl-3-phenyl-1-prop-2-enyloxycarbonyl (Mspoc); see, e.g., Carpino et al., The 2-methylsulfonyl-3-phenyl-1-prop-2-enyloxycarbonyl (Mspoc) Amino Protecting Group, J. Org. Chem. 1999, 64, 8399-8401.

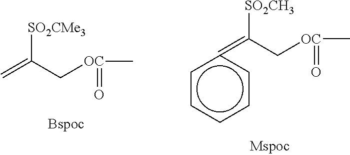

As a general point, the basic aspects of SPPS are generally well-understood in the art and by the skilled person. Thus, they will not necessarily be repeated in detail herein. Such aspects include the choice of resin and resin characteristics, and these are familiar to the skilled person, who can select an appropriate resin from among the available commercial choices and without undue experimentation.

It will be understood that one of the advantages of the invention is the capability to use only water, only alcohol, or only a water-alcohol mixture; i.e., without other solubility-enhancing additives.

It will also be understood that the choice of solvent as between and among water, alcohol, and water-alcohol mixtures (as well as the water:alcohol ratio of any given mixture) will depend to some greater or lesser extent upon the amino acids desired for the target peptide, or the base selected for deprotection, or a combination of these factors. The straightforward nature of the invention enables the skilled person to make the selection on a case-by-case basis and without undue experimentation.

In exemplary embodiments, the method can also include irradiating the acid and the solvent with microwaves during the deprotection step. A detailed description of an instrument suitable for microwave irradiation is the SPPS context is set forth in commonly-owned U.S. Pat. No. 7,393,920 (and in a number or related patents and published applications), the contents of which are incorporated entirely herein by reference.

Typically, the deprotection is carried out using a base that is soluble in the water, alcohol or mixture solvent system. In exemplary embodiments, the base can be selected from the group consisting of, sodium hydroxide, lithium hydroxide, sodium carbonate, piperazine, piperidine, 4-(Amino methyl)piperidine (AMP) and other alkyl (e.g., C₁-C₃) hydroxides. In general, the solubility of simple organic bases (such as amines) is similar to that of simple alcohols. Thus, amines with one to three carbon atoms may be appropriate. Other soluble amines (e.g. piperizine) are also appropriate in many circumstances.

In exemplary embodiments, the protected amino acid is one of the essential amino acids that remain water-soluble when protected with the relevant protecting group; e.g. with Bsmoc. In this embodiment water is used as a solvent and a base that is soluble in water is used in an amount and to the extent necessary to deprotect the acid. It will be understood that the solubility of certain organic bases may limit the amount that can be used in the water, alcohol or mixture solvent, but that a base is acceptable provided that a sufficient proportion is soluble in the solvent system to carry out the desired deprotection.

The method can further comprise washing the deprotected acid in a solvent selected from the group consisting of water, alcohol, and mixtures of water and alcohol. Thereafter, the washed deprotected acid can be coupled to a resin-based peptide or to a resin-based amino acid, again in a solvent selected from the group consisting of water, alcohol, and mixtures of water and alcohol.

The coupled composition can then be washed in the same solvent system; i.e. water, or alcohol, or mixtures of water and alcohol.

In accordance with appropriate peptide synthesis, the method can comprise repeating the steps of deprotecting, washing, coupling, and washing for a second protected acid. Thereafter, the steps can be repeated to add a third protected amino acid, and thereafter a successive plurality of protected amino acids to produce a desired peptide.

When the deprotection step is carried out in a mixture of water and alcohol, any alcohol is appropriate that is miscible with water and that does not otherwise interfere with the ongoing reactions or with the starting materials or the intermediate or final peptide chains. In most circumstances, the alcohol can be selected from the group consisting of methanol, ethanol, 1-propanol, 2-propanol, n-butanol, isobutanol, sec-butanol and tert-butanol. Generally, alcohols with five or more carbons tend to behave like hydrocarbons and are immiscible in water.

In another aspect, and potentially independent of the deprotection step, the invention is a method of solid phase peptide synthesis in which the improvement includes the steps of deprotecting an amino group in its protected form that is protected with a protecting group containing a Michael acceptor site composed of an α,β-unsaturated sulfone, and then washing the deprotected acid in a solvent selected from the group consisting of water, alcohol, and mixtures of water and alcohol. In this embodiment, the advantages of the water or alcohol or mixture solvent system can be used for the washing step independently of whether or not the solvent system is used for the deprotection step.

In exemplary embodiments the acid is protected with a protecting group selected from the group consisting Bsmoc, Nsmoc, Bspoc and Mspoc, with a Bsmoc-protected amino acid being most typical.

As in the case of the deprotection step, the washing step can be carried out in the presence of microwave irradiation on an as-needed or as-desired basis. When the washing step is carried out in the mixture of water and alcohol the alcohol again can be selected from the group consisting of methanol, ethanol, 1-propanol, 2-propanol, n-butanol, isobutanol, sec-butanol, and tert-butanol.

In yet another aspect, and independent of the deprotecting and first washing steps, the invention can include the step of coupling an amino group in its protected form that is protected with a protecting group containing a Michael acceptor site composed of an α,β-unsaturated sulfone and has been deprotected, to a resin-based peptide or a resin-based amino acid in a solvent selected from the group consisting of water, alcohol and mixtures of water and alcohol. The coupling step can be carried out under the application of microwave irradiation as may be desired or necessary. When a mixture of alcohol and water is used, the previously-identified alcohols are among those that are most appropriate.

As in other embodiments, in this embodiment the acid is protected with a protecting group selected from the group consisting Bsmoc, Nsmoc, Bspoc and Mspoc, with a Bsmoc-protected acid being exemplary.

Similarly, this coupling step is entirely consistent with carrying out the deprotection step in the water, alcohol or mixture solvent system using the bases identified previously.

In yet another aspect, the invention is a method of solid phase peptide synthesis comprising deprotecting an amino group in its protected form that is protected with a protecting group containing a Michael acceptor site composed of an α,β-unsaturated sulfone, coupling the deprotected acid to a resin-based peptide or a resin-based amino acid, and then washing the coupled composition in a solvent selected from the group consisting of water, alcohol, and mixtures of water and alcohol. As was true with respect to the other steps in the process, the use of the water, alcohol or mixture solvent system can be in some cases limited to the step of washing the coupled composition and does not required that the deprotection or the coupling steps themselves be carried out in the same solvent system.

Bsmoc, Nsmoc, Bspoc and Mspoc protected amino acids are again exemplary.

Indeed, each of the steps can be carried out in any one or more of the solvent systems or even a nonaqueous solvent system as may be desired or necessary.

The step of washing the coupled composition can likewise be enhanced in some circumstances by the use of microwave irradiation. The alcohols used for the water-alcohol mixture solvent system can be those mentioned previously and the bases used to deprotect the protected amino acids can be those bases named previously.

In another aspect, the invention is a solid phase peptide synthesis method that includes the following steps: deprotecting an amino group in its protected form that is protected with a protecting group containing a Michael acceptor site composed of an α,β-unsaturated sulfone in a solvent system selected from the group consisting of water, alcohol, and mixtures of water and alcohol; washing the deprotected acid in a solvent selected from the group consisting of water, alcohol, and mixtures of water and alcohol; coupling the deprotected acid to a resin-based peptide or a resin-based amino acid in a solvent selected from the group consisting of water, alcohol, and mixtures of water and alcohol; and washing the coupled composition in a solvent selected from the group consisting of water, alcohol, and mixtures of water and alcohol.

As in other embodiments, Bsmoc, Nsmoc, Bspoc and Mspoc protected amino acids are again exemplary.

In order to enhance the reaction, microwaves can be applied during the deprotection step or the coupling step, including the steps of coupling single acids together or the step of coupling a sequential acid to a resin-based peptide or a resin based amino acid.

As in the previous embodiments, appropriate alcohols can include methanol, ethanol, 1-propanol, 2-propanol, n-butanol, isobutanol, sec-butanol, and tert-butanol.

Any appropriate base can be used to deprotect the relevant amino acids, but in exemplary embodiments, including Bsmoc-protected acids, the bases are selected from among mild alkyl (e.g., C₁-C₃) hydroxide bases, sodium hydroxide, lithium hydroxide, sodium carbonate, piperidine, 4-(Amino methyl)piperidine and piperizine.

The deprotecting, coupling and washing steps can be repeated to add a second amino acid that is likewise initially protected with Bsmoc to the first amino acid. The steps can be repeated for a third and thereafter successive plurality of Bsmoc-protected acids to form a peptide chain.

The method can further include the step of cleaving the peptide chain from the solid phase resin, and microwave radiation can be applied to enhance the cleaving step.

In another aspect, the invention is a composition. In this aspect, the composition comprises a mixture of a solid phase resin and a solution. The solution includes a mixture of a solid phase resin and a solution. The solution comprises an amino acid and an amino acid protecting group, both dissolved in the same solvent. The protecting group acts as—i.e., includes the relevant functional group or groups—a Michael Reaction acceptor in the presence of a Michael Reaction donor. The solvent is selected from the group consisting of water, alcohol, and mixtures of water and alcohol.

In exemplary embodiments, the composition further comprises a base that is soluble in the solvent system. In particular embodiments, the base is soluble in water alone. Water soluble bases appropriate for the composition include mild alkyl hydroxide bases, sodium hydroxide, lithium hydroxide, sodium carbonate, piperidine, 4-(Amino methyl)piperidine and piperazine.

In exemplary embodiments, Bsmoc (or Nsmoc, Bspoc or Mspoc) and an amino acid are dissolved in the same solvent.

The alcohol in the composition can in exemplary embodiments be selected from the group consisting of methanol, ethanol, 1-propanol, 2-propanol, n-butanol, isobutanol, sec-butanol, and tert-butanol.

In some embodiments, the deprotection can be carried out in the presence of a sufficient amount of detergent to render the protected acid soluble in the aqueous-based solvent system. The term “soluble” is used herein in its usual sense; i.e., the desired or necessary amount of protected acid will completely dissolve in the solvent system. Persons of ordinary skill in the chemical arts will recognize, of course, that solubility is a relative term that can also be quantified based on the amount of a particular material that will dissolve in a particular solvent. Thus, for purposes of the invention, the respective compositions are considered soluble if they will dissolve in water in the amounts typically required to successfully carry out solid phase peptide synthesis.

Because the progress of deprotection reactions are typically monitored on a periodic sample basis using an ultraviolet measurement of the amount of protecting group in solution, the detergent should avoid interfering with the UV absorption of the protecting group at the wavelengths characteristic of the protecting group.

Detergents are water soluble molecules classified according to their hydrophilic or hydrophobic character (or the degree of each) and their ionic groups. These characteristics establish the behavior of the detergent with respect to the protecting groups, the peptide chain, and individual amino acids.

In many cases a detergent has a hydrophobic tail that associates to form micelles, or that aggregates, or interacts with other molecules (lipids, proteins). In solution, detergents help keep molecules in solution by dissociating aggregates, and unfolding larger molecules

Typical detergents that are helpful include nonionic detergents, cationic detergents, anionic detergents, and zwitterionic detergents. Particular detergents that are useful include octyl phenyl ethylene oxide; sodium lauryl sulfate; and sodium dodecyl sulfate.

In a manner consistent with conventional SPPS, the method can include activating the deprotected acid with an activator that is soluble in the aqueous solvent system. Any activator that carries out the appropriate advantages (i.e. making the oxygen a better leaving group) and that otherwise is consistent with the overall SPPS reaction is appropriate. Representative activating agents include water soluble carbodiimides and triazoles. Other conventional activating agents can include O-Benzotriazolyl-N,N,N′,N′-tetramethyluronium hexafluorophospate (HBTU), 2-(1H-Benzotriazole-1-yl)-1,1,3,3-Tetramethyluronium Tetrafluoro Borate (TBTU), Boc-histidine(tosyl); BOP and BOP-Cl.

In yet another aspect, the invention is a process for accelerating the solid phase synthesis of peptides. In this embodiment, the invention comprises deprotecting the alpha amino group of a an amino group in its protected form that is protected with a protecting group containing a Michael acceptor site composed of an α,β-unsaturated sulfone and linked to solid phase resin particles by admixing the protected linked acid with a deprotecting solution in a microwave transparent vessel while irradiating the admixed acid and solution with microwaves. The method includes activating a second amino acid and then coupling the second amino acid to the first amino acid while irradiating the composition in the same vessel with microwaves. Thereafter the method includes successively deprotecting, activating, and coupling a plurality of amino acids into a peptide without removing the peptide from the same vessel between cycles.

In exemplary embodiments, the amino acid is protected with Bsmoc, Nsmoc, Bspoc or Mspoc.

An instrument suitable for use in the method is described in detail in commonly assigned U.S. Pat. No. 7,393,920. The same description is set forth in other commonly assigned U.S. patents resulting from divisional and continuing applications and has also been published in Europe, for example at EP 1 491 552 and EP 1 923 396. These descriptions provide the skilled person with the information helpful to practicing the method.

The method can further comprise cooling the vessel during any one or more of the deprotecting, activating, and coupling steps to prevent heat accumulation from the microwave energy from degrading the solid phase support or the peptide.

The method can comprise cyclically repeating the steps of deprotecting, activating, and coupling for three or more amino acids in succession to thereby synthesize a desired peptide.

In particular, and in a manner congruent with the steps described in U.S. Pat. No. 7,393,920, the method comprises carrying out the successive deprotecting, activating, coupling and cleaving steps in the single vessel without removing the peptide or the solid phase resin from the vessel between cycles.

The mixture can be agitated with nitrogen or another appropriate inert gas during one or more of the deprotecting, activating, coupling and cleaving steps. In many circumstances, the method will further comprise deprotecting a side chain of the amino acid and in some cases, the side chain will be protected with a T-butanol-based side chain protecting group. Accordingly, the side chain will be deprotected with a composition suitable for that purpose.

When the peptide (intended or desired) is complete, any of the methods described herein typically comprises cleaving the linked peptide from the solid phase resin by admixing the link peptide with the cleaving composition. In some embodiments cleavage is carried out in the same vessel while irradiating the composition with microwaves.

As recognized by the skilled person, the cleaving compositions and protocol are to some extent dictated by the amino acids in the peptide chain and in some cases by the side protecting groups that those amino acids may carry. In most cases, an acid is used to carry out the cleaving step. In general, the acid should carry out the necessary cleavage without adversely affecting or interfering with the desired peptide and any desired groups (e.g., side chain protecting groups) that are attached to the amino acids in the peptide.

Trifluoroacetic acid and hydrofluoric acid (HF) are common cleaving agents, but are often mixed with small proportions of complementary compositions such as water, phenol and ethanedithiol (EDT). Trifluoromethane sulfonic acid (TFMSA) or trimethylsilyltrifluoromethanesulfonate (TMSOTf) are used as cleaving agents in some cases. These are, of course, exemplary rather than limiting of the cleaving composition possibilities. The cleaved peptide (in solution) can be separated from the cleaved resin by filtration and the peptide can then be recovered from the filtrate by a conventional step such as evaporation or solvent-driven precipitation.

Cleavage is typically carried out in the presence of scavenger compositions (e.g., water, phenol, EDT) which protect the peptide from undesired side reactions during and after the cleaving step. As recognized by the skilled person, the scavengers are generally selected based upon the protecting groups that are present. Thus, the selection is to some extent customized by the skilled person, who can select the appropriate scavengers without undue experimentation.

As in other embodiments described herein, the method can comprise deprotecting the first amino acid (or any of the succeeding amino acids) in a solvent selected from the group consisting of water, alcohol and mixtures of water and alcohol. When mixtures of water and alcohol are used as the solvent, the alcohol can be selected from the group consisting of methanol, ethanol, 1-propanol, 2-propanol, n-propanol, isobutanol, sec-butanol, and tert-butanol.

As in the previously described embodiments, the deprotection step can be carried out using a base that is soluble in the appropriate solvent system. In nonaqueous solvent systems the base can include (examples) and in the aqueous or water-alcohol mixture solvent systems, the base is selected from the group consisting of mild alkyl hydroxide bases, sodium hydroxide, lithium hydroxide, sodium carbonate, piperidine, 4-(Amino methyl)piperidine and piperazine.

Synthesis of Bsmoc

Bsmoc is synthesized from commercially available 1-benzothiophene through hydroxymethylation followed by peracid oxidation. The starting material 1-benzothiophene is readily available at modest pricing.

Elimination Vs. Michael Addition Mechanism

In the method of the invention, the protecting group (e.g., Bsmoc) is removed by a Michael Addition mechanism from a secondary amine. As noted previously, a Michael Addition reaction is the nucleophilic addition of a nucleophile to an alpha, beta unsaturated carbonyl compound. The nucleophile is the Michael Donor (e.g., piperidine) and the alpha, beta unsaturated carbonyl compound is the Michael Acceptor (e.g. an alkene).

The protecting groups developed by Carpino (Bsmoc, Mspoc, Bspoc, Nsmoc) contain a Michael Acceptor group. The Michael Acceptor group for these compounds is an activated alkene group. A Michael Donor (typically a base such as piperidine or piperazine) initiates the reaction and forms a Michael Adduct with the protecting group. Formation of the Michael Adduct leads to an intramolecular rearrangement that cleaves the protecting group from the amino acid.

In the Michael Addition mechanism the deprotection also serves as the scavenging action so that no reactive intermediate is present to react with the free amine group. The Bsmoc group is also more reactive to attack by secondary amines than the Fmoc group. These two factors lower the necessary base needed in the deprotection reaction with Bsmoc protection. This is valuable for minimizing base catalyzed side reactions during deprotection, reducing reagent costs, and lowering waste toxicity.

Enhanced Water Solubility

As compared to Fmoc, the structure of Bsmoc appears more soluble in water based upon its heterocyclic 5-membered ring that has an SO₂ group present. Bsmoc appears to be more soluble because it contains only one additional six-membered carbon ring. A comparison between an Fmoc and Bsmoc compound has been observed in rapid solution phase synthesis. In this type of synthesis, TAEA (tris(2-aminoethyl)amine) is used for deprotection and its adduct with Bsmoc is soluble in water, while its adduct with Fmoc is not.

The potential water soluble methods for the Bsmoc reagent can be performed with or without assistance of microwave energy.

Monitoring Capabilities of Bsmoc

The sulfone-containing protecting groups described herein (e.g., Bsmoc) present opportunities for monitoring after completion of either or both of the deprotection and coupling reactions. The single SO₂ group in these compounds is unique to other reagents used during the step-wise assembly of the peptide. This SO₂ group can be monitored by infrared radiation (IR) to determine the quantitative amounts of Bsmoc (or Nsmoc, Bspoc or Mspoc) present at the end of each reaction. Evidence of the SO₂ group can be used to determine an incomplete removal of Bsmoc at the end of the deprotection. This is advantageous to the UV approach in that it does not require performing the reaction twice to make a comparison.

The coupling reaction can be monitored by IR absorption in two possible ways. The first method is to determine the IR absorption immediately after addition of the amino acid and activator reagents. This provides a baseline for total Bsmoc (Nsmoc, Bspoc, Mspoc) in the reaction vessel at the user defined excess. At the conclusion of the coupling reaction and subsequent washing the IR absorption is then again determined and compared to the initial value (addition of pure solvent in identical volume to amino acid activator solution may be necessary for comparison). A 100% complete coupling reaction should yield an IR absorption ratio that is proportional to the excess used. This approach is advantageous because it only requires the coupling reaction to be performed one time. A second approach could make a comparison of the IR absorption after two subsequent coupling reactions in a manner identical to that currently used by UV for monitoring the Fmoc deprotection step.

The skilled person will understand that the invention includes numerous possibilities, any of which can be carried out by the skilled person and without undue experimentation. Thus, the deprotection can be carried out using amino acids protected with the Michael addition acceptor compounds, including, but not limited to Bsmoc, Nsmoc, Bspoc and Mspoc. Any one or more (or all) of the deprotection, washing, activation, coupling or cleaving steps can be carried out in water or in a water-alcohol system, with or without a detergent. Any one or more (or all) of these steps can likewise be enhanced by applying microwave irradiation.

In the specification there have been set forth preferred embodiments of the invention, and although specific terms have been employed, they are used in a generic and descriptive sense only and not for purposes of limitation, the scope of the invention being defined in the claims.

1. In a solid phase peptide synthesis method, the improvement comprising
deprotecting an amino group in its protected form that is protected with a protecting group containing a Michael acceptor site composed of an α,β-unsaturated sulfone; and
washing the deprotected acid in a solvent selected from the group consisting of water, alcohol, and mixtures of water and alcohol.

2. A method according to claim 1 wherein the protecting group is selected from the group consisting of Bsmoc, Nsmoc, Bspoc and Mspoc.

3. A method according to claim 1 in which the protecting group is Bsmoc and the washing solvent is water.

4. A method according to claim 1 further comprising irradiating the deprotected acid and the solvent with microwave irradiation during the washing step.

5. A method according to claim 1 comprising deprotecting the protected acid with a base that is soluble in the solvent.

6. A method according to claim 1 wherein the washing step is carried out in a mixture of water and alcohol and wherein the alcohol is selected from the group consisting of methanol, ethanol, 1-propanol, 2-propanol, n-butanol, isobutanol, sec-butanol, and tert-butanol.

7. A method according to claim 1 comprising deprotecting the protected amino acid with a base selected from the group consisting of sodium hydroxide, lithium hydroxide, sodium carbonate, piperidine, 4-(Amino methyl)piperidine, piperazine and alkyl hydroxides.

8. A method according to claim 1 comprising coupling the washed deprotected acid to a resin-based peptide or a resin-based amino acid in a solvent selected from the group consisting of water, alcohol, and mixtures of water and alcohol.

9. A method according to claim 8 comprising repeating the steps of:
deprotecting;
washing;
coupling; and
washing;
for a second protected acid.

10. In a solid phase peptide synthesis method, the improvement comprising:
deprotecting an amino acid that is protected with a protecting group that contains a Michael acceptor site composed of an α,β-unsaturated sulfone;
in a solvent selected from the group consisting of water, alcohol and mixtures of water and alcohol.

11. A method according to claim 10 wherein the protecting group is selected from the group consisting of Bsmoc, Nsmoc, Bspoc and Mspoc.

12. A method according to claim 10 wherein the protecting group is Bsmoc and the deprotection solvent is water.

13. A method according to claim 10 further comprising irradiating the acid and the solvent with microwaves during the deprotection step.

14. A method according to claim 10 comprising deprotecting the Bsmoc-protected acid with a base that is soluble in the solvent.

15. A method according to claim 10 comprising deprotecting the Bsmoc-protected acid with a base selected from the group consisting of sodium hydroxide, lithium hydroxide, sodium carbonate, piperidine, 4-(Amino methyl)piperidine, piperazine and alkyl hydroxides.

16. A method according to claim 10 comprising coupling the deprotected acid to a resin-based peptide or a resin-based amino acid in a solvent selected from the group consisting of water, alcohol, and mixtures of water and alcohol.

17. A method according to claim 10 comprising repeating the steps for a third and thereafter successive plurality of protected amino acids.

18. A method according to claim 10 wherein the deprotection step is carried out in a mixture of water and alcohol, and the alcohol is selected from the group consisting of methanol, ethanol, 1-propanol, 2-propanol, n-butanol, isobutanol, sec-butanol, and tert-butanol.

19. In a solid phase peptide synthesis method, the improvement comprising:
deprotecting an amino group in its protected form that is protected with a protecting group containing a Michael acceptor site composed of an α,β-unsaturated sulfone; and
coupling the deprotected acid to a resin-based peptide or a resin-based amino acid in a solvent selected from the group consisting of water, alcohol, and mixtures of water and alcohol.

20. A method according to claim 19 wherein the protecting group is selected from the group consisting of Bsmoc, Nsmoc, Bspoc and Mspoc.

21. A method according to claim 19 wherein the protecting group is Bsmoc and the coupling solvent is water.

22. A method according to claim 19 further comprising irradiating the deprotected acid and the solvent with microwave irradiation during the coupling step.

23. A method according to claim 19 wherein the coupling step is carried out in water or a mixture of water and alcohol the alcohol is selected from the group consisting of methanol, ethanol, 1-propanol, 2-propanol, n-butanol, isobutanol, sec-butanol, and tert-butanol.

24. A method according to claim 19 comprising deprotecting the protected acid with a base selected from the group consisting of sodium hydroxide, lithium hydroxide, sodium carbonate, piperidine, 4-(Amino methyl)piperidine, piperazine and alkyl hydroxides.

25. A method according to claim 19 comprising irradiating the protected amino acid and the solvent with microwaves during the deprotection step.

26. A method according to claim 19 comprising repeating the deprotecting and coupling steps for a third and thereafter successive plurality of protected acids to form a peptide chain.

27. A method according to claim 26 comprising cleaving the peptide chain from the solid phase resin.

28. A method according to claim 27 comprising irradiating the composition with microwaves during the cleaving step.

29. A composition comprising:
a mixture of a solid phase resin and a solution; wherein
said solution comprises an amino acid and an amino acid protecting group, both dissolved in the same solvent;
said protecting group contains a Michael acceptor site composed of an α,β-unsaturated sulfone; and
said solvent is selected from the group consisting of water, alcohol, and mixtures of water and alcohol.

30. A composition according to claim 29 wherein said protecting group is selected from the group consisting of Bsmoc, Nsmoc, Bspoc, and Mspoc.

31. A composition according to claim 29 further comprising a water soluble base.

32. A composition according to claim 31 wherein said water soluble base is selected from the group consisting of sodium hydroxide, lithium hydroxide, sodium carbonate, piperidine, 4-(Amino methyl)piperidine, piperizine and alkyl hydroxides.

33. A composition according to claim 29 wherein said solvent is a mixture of alcohol and water and said alcohol is selected from the group consisting of methanol, ethanol, 1-propanol, 2-propanol, n-butanol, isobutanol, sec-butanol, and tert-butanol.
